(19) United States
(12) Patent Application Publication
(10) Pub. No.: US 2019/0314184 A1
(43) Pub. Date: Oct. 17, 2019

(54) Medical Rehabilitation Boot And Method For Preventing Heel Decubitus

(71) Applicant: Jeffrey W Gainey, Peachtree City, GA (US)

(72) Inventor: Jeffrey W Gainey, Peachtree City, GA (US)

(21) Appl. No.: 15/953,067

(22) Filed: Apr. 13, 2018

(51) Int. Cl. A61F 5/01; A61F 13/06; A61F 5/30

(52) U.S. Cl. CPC A61F 5/0111; A61F 5/30; A61F 5/0195; A61F 13/069

(57) ABSTRACT

Disclosed is a medical rehabilitation boot, comprising a unitary, foldable body form further comprising five individually-stitched sections, or pockets, each filled with either fiber or foam. The pockets are formed by custom-located stitches which form seams, the seams collectively joining an inner fabric and an outer fabric around the perimeter of the boot. The five sections consist of a left sidewall further containing an upper and a lower tube-hole, a right sidewall also containing an upper and a lower tube-hole, a rectangular bottom section having a heel arc at one end, an inner flap, and an outer flap. A continuous, one-piece outer fabric and a continuous, one-piece inner fabric form pockets of containment for fiber filling in four of the five sections. A sloped foam wedge inserted into the bottom section contributes to the functional comfort of a patient wearing the boot.

### CROSS-REFERENCES TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### BACKGROUND OF THE INVENTION

### (1) Field of the Invention

This invention relates to the prevention or cure of heel sores for bed patients. The product is a medical boot, referred to as the “HeelCheck Boot,” 1 which utilizes a unique combination of angled foam and fiber. The HeelCheck Boot 1 includes several other features that enhance the healing and prevention of heel sores while ensuring no other medical problems are created for the patient.

Heretofore several different foot and leg devices have been proposed for the cure or prevention of heel sores. These sores are known more specifically as decubitus ulcers, and generally form by frictional irritation between the heel of a bed ridden patient and the bed linens or mattress. Lying in a particular position for a prolonged period produces external pressures on skin and tissues covering rigid or bony body parts, such as the heel. This pressure compromises the blood supply to those rigid or bony areas which, after a sustained period, results in “bed sores.”

### (2) Description of the Related Art, Including Information Disclosed under 37 CFR 1.97 and 1.98

U.S. Pat. No. 4,076,022 Therapeutic foot and leg protector—contains a rigid outer shell having a soft, protective liner. The protector is perforated in the heel portion for air circulation.

U.S. Pat. No. 6,256,804 Body Supports and protectors—The pillows and supports are made for supporting various body parts, namely neck, shoulder, torso, elbows, tail bone.

U.S. Pat. No. 5,328,445 Inflatable foot cushion—uses several, movable inflatable foot cushions and also involves the use of a shoe spoon and rigid base.

U.S. Pat. No. D411,758 Ornamental design for a heel protector. Product utilizes foam.

U.S. Pat. No. 5,367,789 is a protective medical boot and orthotic splint—a soft medical boot with splint bar for adjusting angle of the foot. The device comprises a rigid insole, rigid splint bar, hinge internal sac filled with air/water/gel, and a rubberized laminated sole.

U.S. Pat. No. 5,449,339 Heel supporting protective boot for bed patients—foam with foot and leg supporting portions. The invention features friction reducing material and extra padding composed of foam, serving to limit foot drop.

U.S. Pat No. 8,834,396; 16 Sep. 2014; A heel supporting and protective boot for bed-ridden patients, comprising unitary exterior and interior body forms having fiber filling and an angled foam wedge. Sewn tube holes on both sides allow for insertion of medical tubing. The inner fabric of the boot is antibacterial and moisture absorbent, while the outer fabric is moisture repellant. Left and right “Y-straps” provide secure upper and lower fastening. An arc-type opening at the heel of the bottom section of the boot and a circular cutout in the foam wedge allow a patient's heel to overhang freely exposed to air, provide proper elevation, and enhance blood circulation.

### BRIEF SUMMARY OF THE INVENTION

The foregoing objects and advantages of the present invention are accomplished in illustrated embodiments disclosed herein, which embodiments comprise a new and improved heel-supporting protective boot for bed-ridden patients. The boot, referred to as the “HeelCheck” boot 1, has a unique combination of foam and fiber materials fabricated within five interconnected sections of the HeelCheck boot 1.

In essence, the HeelCheck Boot 1 comprises a unitary, foldable body form further comprising five individually-stitched sections, or pockets, each filled with either fiber or foam. The pockets are formed by custom stitches which form seams, joining an inner fabric 5 and an outer fabric 4. The five sections consist of a left sidewall 10 further containing an upper and a lower tube-hole 14, 15, a right sidewall 20 also containing an upper and a lower tube-hole 25, 26, a rectangular bottom section 33 having a heel arc 32 at one end, an inner flap 23, and an outer flap 13. A continuous, one-piece outer fabric 4 and a continuous, one-piece inner fabric 5, and a sloped foam wedge 40 contribute to the functional qualities of the five sections.

Of the above five sections, the inner flap 23 manifests a foam 3 filling, and the bottom section 33 contains a sloped, rectangular foam wedge 40 which foam wedge 40 is further enveloped with a surrounding quantity of fiber 2 fill. The remaining three sections are filled entirely with fiber fill 2. Individual stitching 8 is performed at specific, corresponding locations in the unfolded body forms of both the outer fabric 4 and the inner fabric 5, thereby resulting in the above-described sections, or pockets of the HeelCheck Boot 1. All five sections, in functional use, are folded into a shape resembling the contours of the human foot, and a series of cross-straps and Y-shaped straps and appropriate connecting means serve to maintain the HeelCheck Boot 1 securely about the foot 51 of a patient.

### OBJECTIVES OF THE INVENTIVE CONCEPT

It is a primary objective of the present inventive concept to provide a new and improved heel boot for bed-ridden patients which eliminates many of the disadvantages and problems encountered with prior art medical boots.

It is another object of the present inventive concept is to design a new and improved heel supporting boot with the features herein described which is especially useful in the prevention or cure of bed sores on the heel.

Another objective of the HeelCheck Boot 1 is to eliminate friction and pressure contact between a patient's heel and the bedding surface.

A further object of the inventive concept to provide an improved method of elevating a patient's the heel without hyper-extending the knee. Adequate elevation also facilitates the exposure of the heel to a continuous circulation of air.

Another objective of the inventive concept is to provide sewn tube holes in the left and right sidewalls of the HeelCheck Boot 1 so that a sequential compression pump or other necessary medical tubing can be used 1 with the boot.

An important object of the inventive concept is to utilize the cushioning effect of a combination of fiber filling and an angled foam wedge to facilitate the healing and/or prevention of decubitis ulcers of the heel.

Another objective of the HeelCheck Boot 1 is to furnish to hospitals and caregivers a new medical boot that does not allow bacteria to grow inside the boot.

A further object of the inventive concept is to construct a product that utilizes moisture absorbing fabric on the interior surfaces of the medical boot and additionally, provide a moisture repelling fabric on the exterior surfaces of the boot.

An important objective of the inventive concept is to provide a new method of secure and effective strapping for the purpose of eliminating the possibility of “foot drop” of bed-ridden patients.

A further objective of the inventive concept is to utilize a material that will ensure necessary air ventilation and minimize internal heat buildup.

Another objective of the inventive concept is to utilize a horizontally flat design shape of the foam wedge, to effectively inhibit internal or external rotation of the patient's lower leg, thus avoiding knee and hip joint problems.

An important objective of this inventive concept is to ensure ease of operation of the HeelCheck Boot 1 for the caregiver with respect to both fitting the boot onto a patient as well as removing the boot.

Another objective of the HeelCheck Boot 1 is to construct it so as to effectively eliminate inadvertent disengagement of the patient's foot.

A further object of the inventive concept is to provide an improved boot that does not soil due to patient incontinence.

Another objective of the inventive concept is to ensure that there are no pressure points created within the HeelCheck Boot 1 while at the same time, maintaining adequate elevation of the patient's heel.

Another objective of the inventive concept is to provide a stable, durable boot that ensures effective healing care for patients regardless of their size or weight.

An important objective of the inventive concept is to provide a medical boot which is economical to manufacture and simple to utilize by nursing personnel while accomplishing a multitude of healing goals.

### BRIEF DESCRIPTION OF THE VIEWS OF DRAWINGS AS EXEMPLARY EMBODIMENTS OF THE INVENTIVE CONCEPT

For a better understanding of the invention, reference will be made to the following detailed description taken in conjunction with the drawings, in which:

FIG. 1 is a view looking directly into the front entry of the HeelCheck Boot 1, toward the inner flap 23.

FIG. 2 is an illustration of the right side of the HeelCheck Boot 1, in its fully closed configuration, with the outer flap 13 secured onto the inner flap 23, further showing the right upper and lower Y-straps 27, 28.

FIG. 3 is a left-side view of the HeelCheck Boot 1, in a fully closed configuration, with the outer flap 13 secured onto the inner flap 23, further showing the right upper and lower Y-straps 27, 28.

FIG. 4 is a view of the entirety exterior surfaces/fabric of the unfolded body form of the HeelCheck Boot 1 as shown completely unfastened and laid out.

FIG. 5 presents the entirety interior surfaces/fabric of the unfolded body form of the HeelCheck 1 as shown completely unfastened and laid out.

FIG. 6 is top view of the Heel Check Boot 1 boot, showing the completely fastened upper and lower cross-straps 30, 31 and the outer flap 13 fastened onto the inner flap 23.

FIG. 7 is a cross-sectional view of the HeelCheck Boot 1, as depicted from the perspective of section lines 7-7 shown in FIG. 6.

FIG. 8 is a cross-sectional view of the HeelCheck Boot 1, as depicted from the perspective of section lines 8-8 shown in FIG. 6.

FIG. 9 depicts a stylized view of the position of a patient's foot 51 and lower leg 50 relative to the orientation of the foam wedge 40, which is constructed to fit into the bottom outer section 33 of the HeelCheck Boot 1.

### DETAILED DESCRIPTION OF THE INVENTION

The objects, features, and advantages of the concept presented in this application are more readily understood when referring to the accompanying drawings. The drawings, totaling nine figures, show the basic components of the HeelCheck Boot 1 and the manner in which this inventive concept is used. In the several figures, like reference numbers are used in each figure to correspond to the same component as may be depicted in other figures.

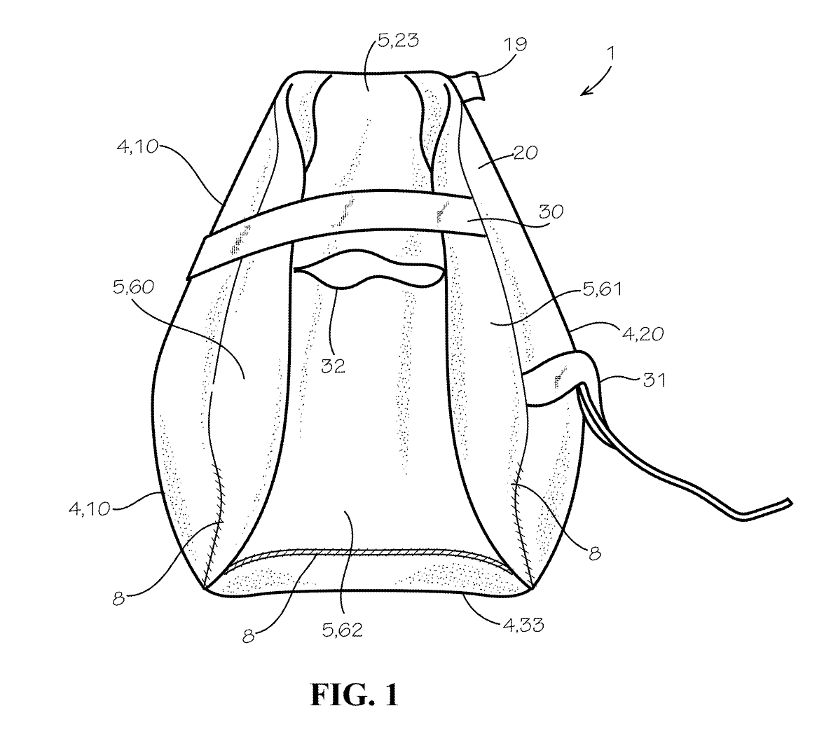

Referring now more particularly to the drawings herein, and beginning with FIG. 1, where there is shown a view of the front opening to the HeelCheck Boot 1, looking toward the rear of the HeelCheck Boot 1. The HeelCheck Boot 1 essentially comprises five sections, being a left sidewall, a right sidewall a bottom section, an outer flap, and an inner flap. Prominent components shown in FIG. 1 are the left outer sidewall 10, the left inner sidewall 60, the right inner sidewall 61, the right outer sidewall 20, and the bottom inner surface 62. Also shown, looking toward the rear of the HeelCheck Boot 1 are the inner flap 23, a fastened upper cross-strap 30, and an un-fastened lower cross-strap 31. Further, toward the rear of the HeelCheck Boot 1, a heel arc 32 is visible just below the inner flap 23.

The upper and lower cross-straps 30, 31 must normally be unfastened in order to separate the left and right inner sidewalls 60, 61, thereby allowing insertion of a patient's foot. The bottom of the patient's foot will be proximate to, or abut the inner flap 23, when the foot is fully inserted. The bottom section 33 of the HeelCheck Boot 1 contains a foam wedge 40 (out of view) and is topped by the inner surface 62. A pocket formed by the left outer sidewall 10 and left inner sidewall 60, and a pocket formed by the right inner sidewall 61 and right outer sidewall 20 are each filled with a pliable fibrous material, referred to as “fiber fill” 2. Likewise, a pocket, or section formed by the inner flap 23 also contains fiber fill 2. A section pocket formed by the bottom inner surface 62 and bottom outer surface 63 houses the foam wedge 40, along with a quantity of fiber fill 2 on the top surface of the foam wedge 40.

In the preferred embodiment, the outer surfaces of the HeelCheck Boot 1 comprise a “rip stop” type outer fabric 4, a woven, nylon-threaded material which is generally water resistant. In the preferred embodiment, the inner surfaces of the HeelCheck Boot 1 are fabricated with an inner fabric 5 which may comprise brushed, micro-suede, or similar material that is treated to exhibit anti-bacterial properties. The inner fabric 5 and the outer fabric 4 are shaped into compatible “body forms” and are combined as the result of continuously-stitched 8 junctions, or seams, as illustrated in various locations in the drawing figures.

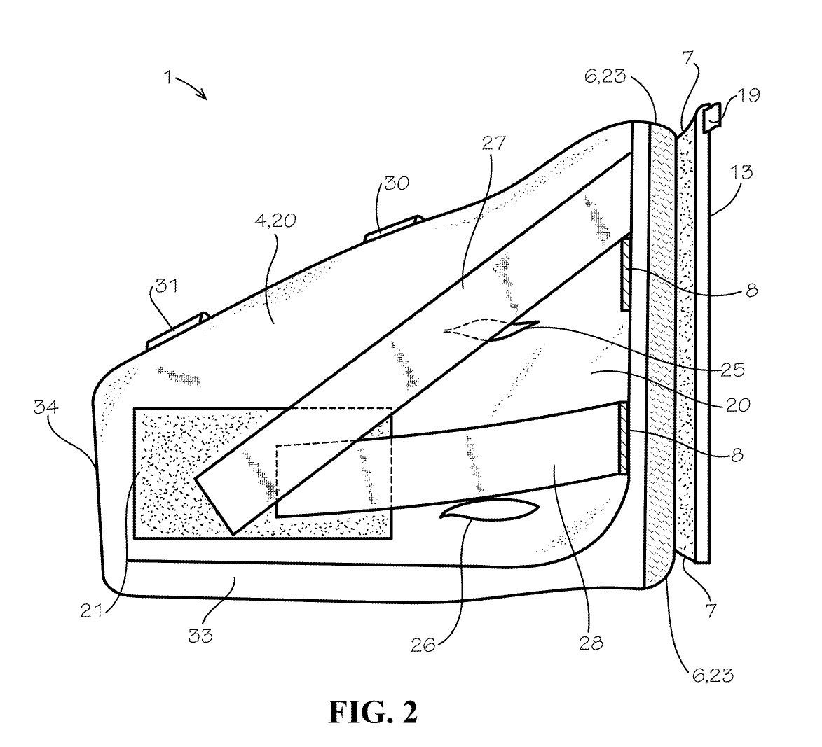

FIG. 2 is a right side view of the HeelCheck Boot 1, prominently showing the right upper Y-strap 27, the right lower Y-strap 28, the rear ends of both Y-straps 27, 28 being stitched 8 onto the rearward portion of the right outer sidewall 20. The Y-straps 27, 28 are designed to fasten onto a right felt connector square 21, shown immediately behind the front section 34 of the HeelCheck Boot 1. In the preferred embodiment, the inner surface of the Y-straps 27, 28 manifest the hook component 6 of a hook-and-felt fastening system, while the right felt connector square 21 consists of the felt component 7 of the hook-and-felt fastening system.

Also depicted in FIG. 2 is a right upper tube hole 25 and a right lower tube hole 26, either of which serves to admit the insertion of medical equipment, or particularly, a tube that may be used with a sequential compression pump. A “TOES” tag 19 is stitched to the upper rear of the outer flap 13, so as to provide visible directions to a caregiver who must make a quick determination as to which end of the HeelCheck Boot 1 the bottom of the patient's foot 51 must be oriented.

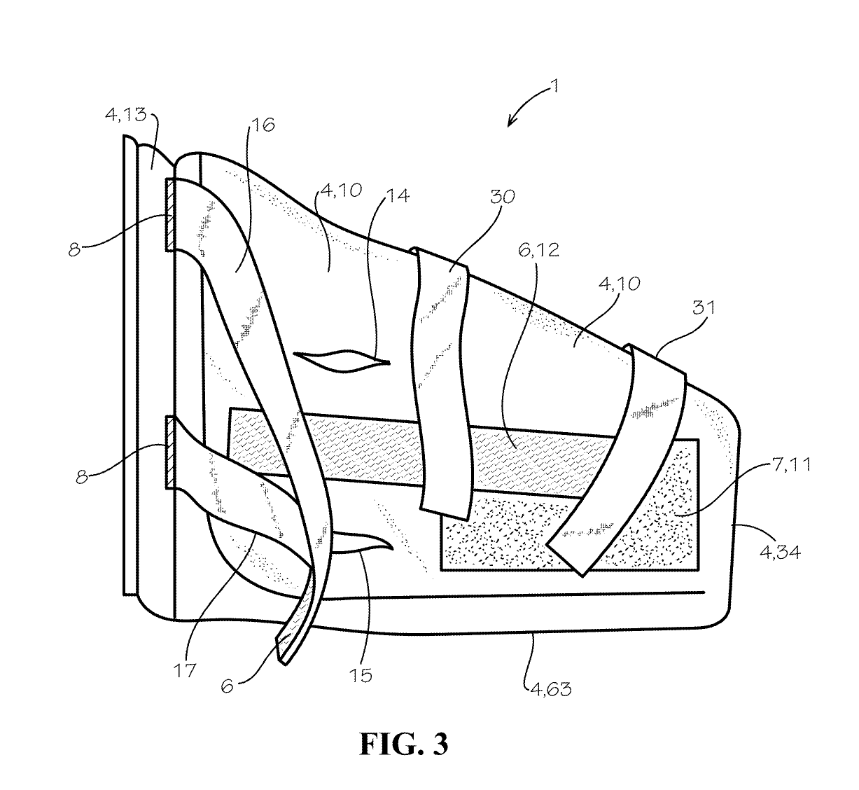

FIG. 3 is a left-side view of the HeelCheck Boot 1, prominently showing the unfastened left upper Y-strap 15 and the left lower Y-strap 17, which are both stitched together near their ends. In the preferred embodiment, when placed in use, the end of the left upper Y-strap 16 is connected by a hook-and-felt fastening mechanism to a left felt connector square 11. Also shown is the upper cross-strap 30, extending across the top of the HeelCheck Boot 1 to fasten onto a left oblong hook fastener 12. A lower cross-strap 31 extends across the lower portion of the HeelCheck Boot 1 to also fasten onto the left oblong hook fastener 12. The left oblong hook fastener 12 comprises the hook 6 component of a hook-and-felt type fastener, while both cross-straps 30, 31, comprise the felt 7 component of a hook-and-felt type fastener.

Further depicted in FIG. 3 are the left upper tube hole 14 and the left lower tube hole 15, both of which serve to admit the insertion of any tube that may be used with required patient treatment. The outer flap 13 is the point of stitched 8 attachment of both the left upper and lower Y-straps 15, 17. The left outer sidewall 10 of the HeelCheck Boot 1 is comprised of a specific outer fabric 4 that also covers the entire outer surfaces of the HeelCheck Boot 1, including the front section 34 and the bottom outer surface 63.

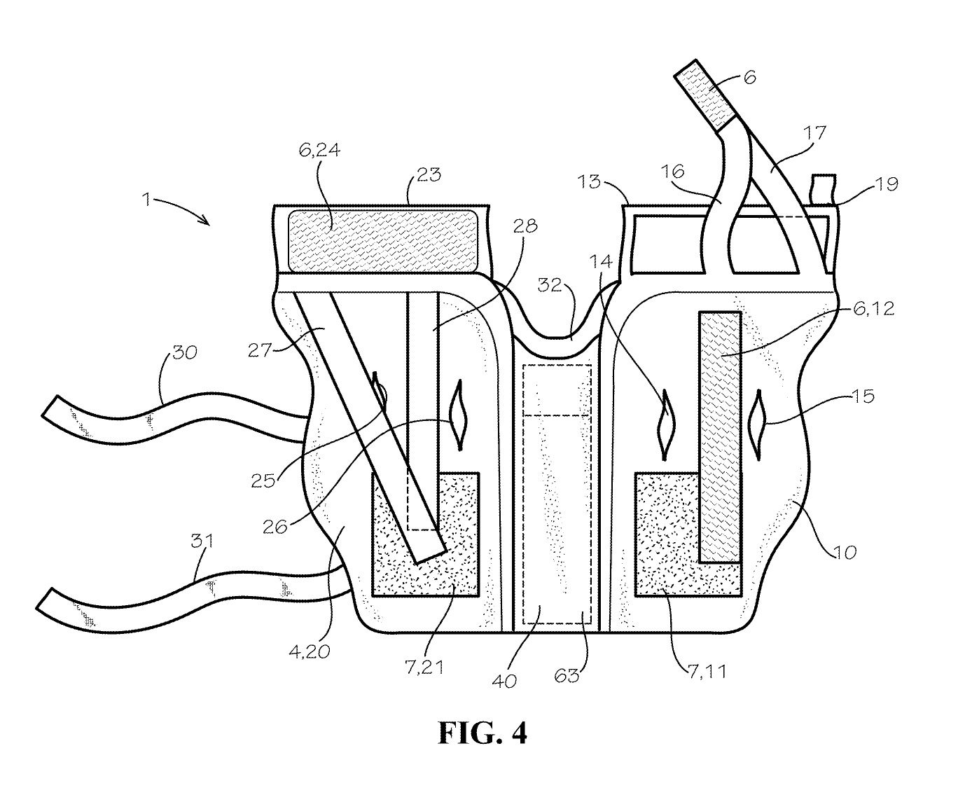

FIG. 4 is the entirety of a “laid-out” view of the unitary exterior body form, or material pattern, of the HeelCheck Boot 1, showing all five of the sections. A dashed outline depicts the relative location of the foam wedge 40 as it would be placed in a pocket encompassed by the bottom outer surface 63 and the bottom inner surface 62 just prior to stitching 8 the inner 5 and outer 4 fabric surfaces of the HeelCheck Boot 1 closed.

Also shown in FIG. 4 are the heel opening 32, the left outer sidewall 10, the right outer sidewall 20, the right upper and lower tube holes 25, 26, the left upper and lower tube holes 15, 14, the right upper and lower Y-straps 27, 28, and the left upper and lower Y-straps 16, 17. FIG. 4 demonstrates that the bottom edge of the right outer sidewall 20 is connected (or stitched) to the right edge of the outer surface 63 of the bottom section, the rear edge of the right outer sidewall 20 is connected to the inner flap 23, the bottom edge of the left outer sidewall 10 is connected to the left edge of the outer surface 63 of the bottom section, and lastly, the rear edge of the left outer sidewall 10 is connected to the outer flap 13.

Further shown in FIG. 4 are the upper and lower cross-straps 30, 31, the right felt connector square 21, left connector square 11, the outer flap 13, and the inner flap 23, which manifests hook 6 component material to enable its connection to the felt 7 fastener component on the inner surface (out of view) of the outer flap 13. The unitary exterior body form, which, in the preferred embodiment, comprises a “rip-stop” type outer fabric 5, is an exact, superimposed image of the unitary interior body form.

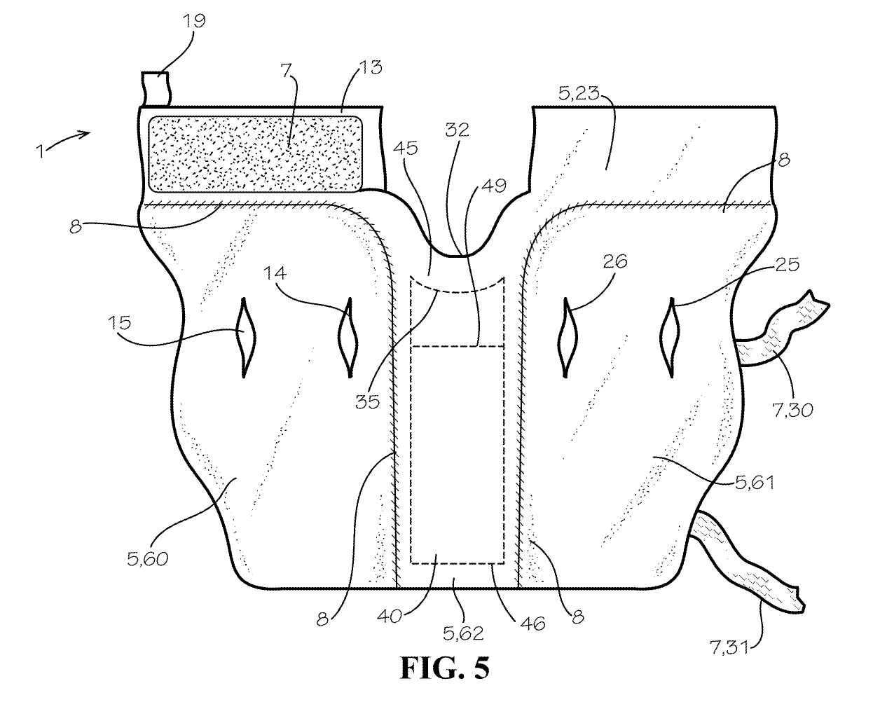

FIG. 5 is a view of the entire “layout” of the unitary interior body form, or material pattern, of the HeelCheck Boot 1. A dashed outline depicts the relative location of the foam wedge 40 as it would be placed under the inner fabric 5 just prior to stitching the HeelCheck Boot 1 closed to form the bottom inner surface 62 of the Heel Check Boot 1. Also shown in FIG. 5 are the heel arc 32, the left inner sidewall 60, the right inner sidewall 61, the left upper and lower tube holes 15, 14, right upper and lower tube holes 25,26, the outer flap 13, and the inner flap 23. The upper and lower cross-straps 30, 31 are shown in their unfastened positions. It is to be noted that the outer flap 13 is filled with foam 3 material, while the inner flap 23 interiorly contains fiber fill 2 material.

The two pairs of tube holes, 14, 15, and 25, 26 may often be required for the insertion of medical tubing. The primary medical tubing that would typically be used in conjunction with a pressure relief medical boot would be a sequential compression pump. Many patients that would require a rehabilitative medical boot, as disclosed herein would probably also need the use of medical tubing. Thus, the left upper and lower tube holes 15, 14, and the right upper and lower tube holes 25, 26 allow for the use of said tubing while still taking advantage of the pressure relief characteristics of the HeelCheck Boot 1.

Also, as visible in FIG. 5, the outer flap 13 features, the felt 7 component of a hook-and-felt fastening system on its outer surface. The inner fabric 5 material of the HeelCheck Boot 1 utilizes a material that has two beneficial features. The first quality is that the inner fabric 5 is antibacterial, meaning it will not allow bacteria to grow and thereby creates an environment hostile to the spread of any strain of bacteria inadvertently introduced into the HeelCheck Boot 1. The second feature of the inner fabric 5 is that it absorbs moisture or other body fluids that may be produced by the patient.

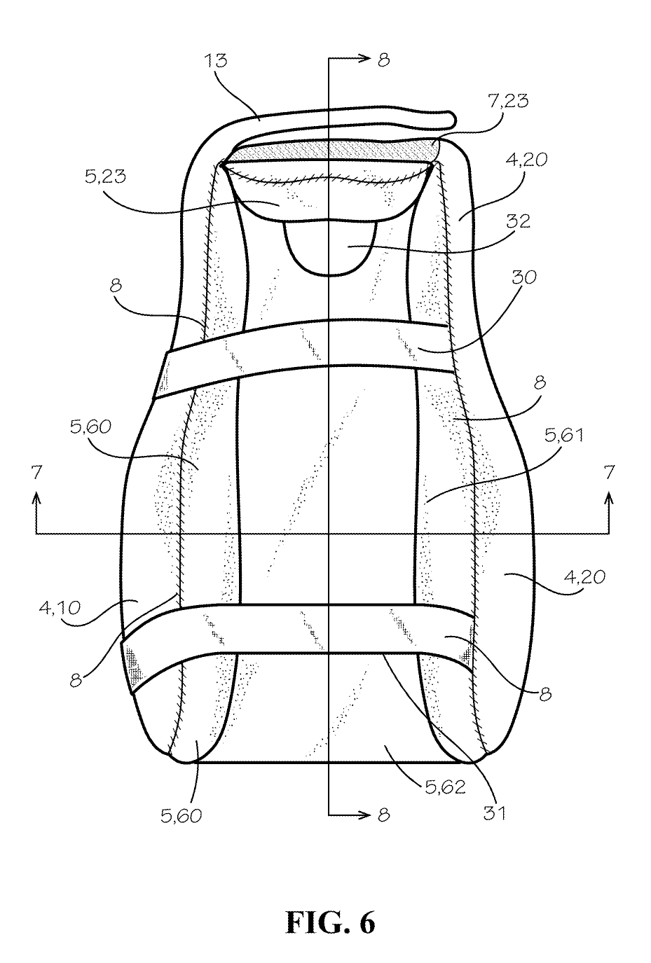

Referring to FIG. 6, there is shown an overhead view of the medical HeelCheck Boot 1. Notable in FIG. 6 are the bottom inner surface 62, the heel arc 32, the left outer and inner sidewalls 10, 60 and the right inner and outer sidewalls 61, 20. The right inner and outer sidewalls 61, 20 and the left inner and outer sidewalls 10, 60 are stitched 8 together forming a seamed pocket which accommodates a quantity of fiber fill 2.

It can be seen that, in functional use of the HeelCheck Boot 1, a patient's lower leg 50 would rest against the bottom inner surface 62 (which forms the upper surface of a pocket containing the foam wedge 40 and an amount of fiber fill 2). Further, the patient's heel 52 would be protruding into the heel arc 32 opening, which serves effectively as a direct means of preventing a heel sore during the time a patient is bedridden. The upper cross-strap 30 and the lower cross-strap 31 serve to retain the patient's lower leg 50 properly within the confines of the Heel Check Boot 1. The bottom of the patient's foot 51 would rest against the inner flap 23, which inner flap 23 would be held in a secure closed position by means of the outer flap 13 securing the inner flap 23 in position.

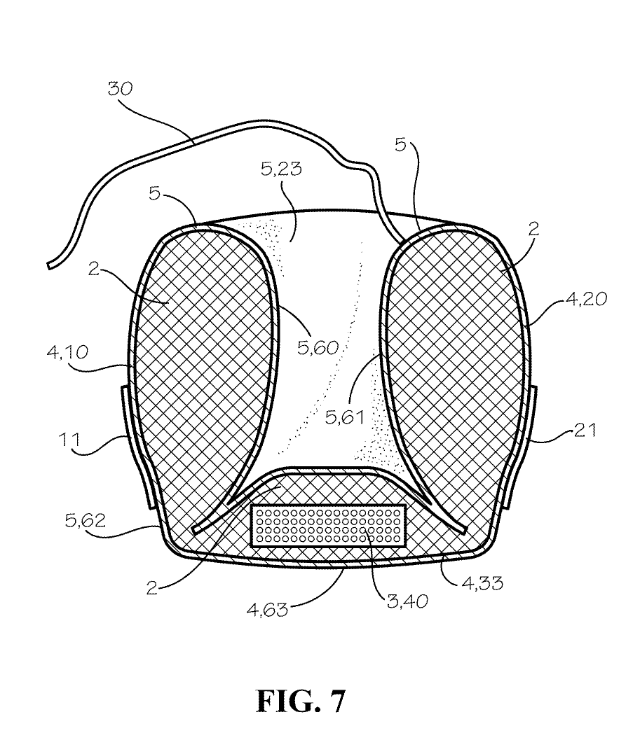

FIG. 7 is a cutaway view of the HeelCheck Boot 1 as seen from the perspective of section line 7-7 in FIG. 6. Most prominent in the cutaway view are the left outer sidewall 10 and left inner sidewall 60, which, when stitched together, form a pocket containing fiber fill 2. Similarly the right inner sidewall 61 and the right outer sidewall 20, when stitched together, form a pocket also containing fiber fill 2. The bottom inner surface 62 and the bottom outer surface 63 form a pocket or section into which is inserted the foam wedge 40, which is then enveloped with in fiber fill 2.

In FIG. 7, it is noted that the foam wedge 2 is composed of a foam material(s) 3 commonly found in industry. As can be seen in FIG. 7, the cross-section of the foam wedge 40 illustrates how the fiber fill 2 material lies upon the top surface of the foam wedge 40, thereby providing a patient with the benefit of cushioning from both the fiber fill 2 and the resilient foam wedge 40. Further discernible in FIG. 7 are the left felt connector square 11, the right felt connector square 21, and the unfastened upper cross-strap 30. The inner surface of the inner flap 23 is further shown.

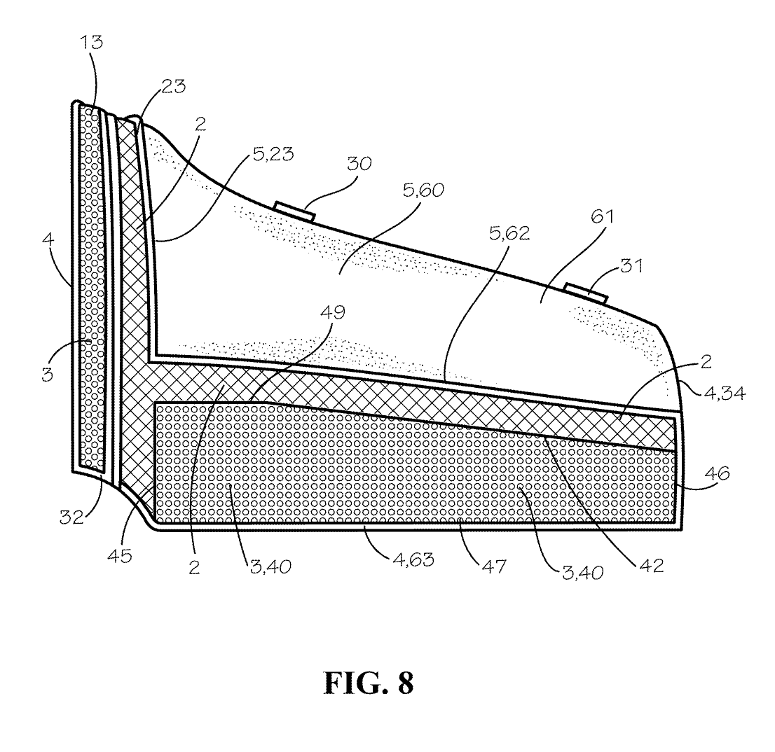

FIG. 8 is a cutaway view of the HeelCheck Boot 1 as seen from the perspective of cutaway line 8-8 in FIG. 6. Most prominent in FIG. 8, it is noted that the foam wedge 40 is composed of a foam material(s) 3 commonly found in industry. As can be seen in FIG. 8, the cross-sectional view illustrates how the fiber fill 2 material lies upon the top surface of the foam wedge 40, thereby providing a patient with the benefit of cushioning from both the fiber fill 2 and the resilient foam wedge 40. Fiber fill 2 is also placed in the interior of the inner flap 23. The outer flap 13 is shown to contain interiorly, foam 3 material.

Further seen in FIG. 8 are the outer flap 13 and its outer fabric 4, the outer flap 13 being attached to the inner flap 23, which inner flap 23 contains fiber fill 2. Also visible is the right inner sidewall 61, the attachment point of the upper cross-strap 30, and the attachment point for the lower cross-strap 31. In FIG. 8, the bottom outer surface 63 comprises a material formed with the outer fabric 4. The heel arc 32 is the open area from which a patient's heel 52 is suspended when lying horizontally on his/her back while wearing the HeelCheck Boot 1. The inner fabric 5 covers all interior surfaces of the HeelCheck Boot 1, while the outer fabric 4 material covers all exterior surfaces of the HeelCheck Boot 1.

The profile view of the foam wedge 40 as shown in FIG. 8 shows that the foam wedge 40 bottom rests atop a thin layer of outer fabric 4, while a layer of fiber fill 2 is packed atop the foam wedge 40. In the fabrication of the HeelCheck Boot 1 the foam/fiber combination provides a unique blending of materials for patient comfort. The fiber fill 2 cushions the bony areas of a patient's lower leg 50 and prevents pressure sores that can be caused by the use of foam support only.

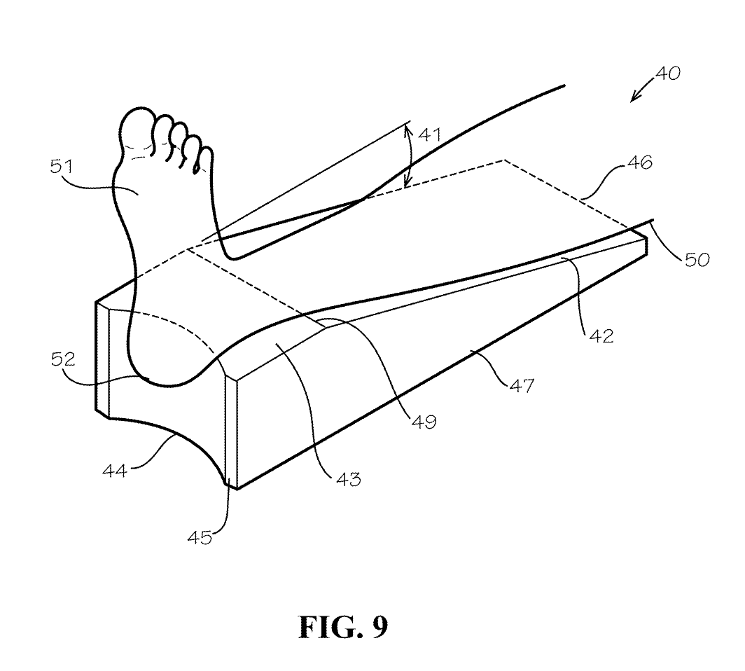

FIG. 9 presents a stylized view of the foam wedge 40 supporting the lower portion of a patient's lower leg 50. The foam wedge 40 is wide enough at its front 46 end so as to extend to the right and left side of a patient's lower leg 50, as depicted in FIG. 9. The foam wedge 40 is constructed with a slope 41 extending upward from the rear end 45 of the foam wedge 40 to a ridge 49 at approximately a thirty degree angle. At the ridge 49, the slope 41 ends and the remaining portion of the top of the foam wedge 40 is flat area, or horizontally level in relationship to a bed surface. The flat area forms the Achilles tendon rest 43 area of the HeelCheck Boot 1, while the majority of the upper surface of the foam wedge 40 provides a leg rest 42 section for a patient's lower leg 50.

The elevation of a patient's foot 51 and clearance of the heel 52 over the circular cutout 44 of the foam wedge 40 helps prevent bed sores, in particular, decubitus of the heel. Blood circulation in the foot 51 is also enhanced. The HeelCheck Boot 1 is constructed of a flat base 47, and of sufficient width, so as to enhance horizontal stability of the HeelCheck Boot 1. The width of the base 47 of the HeelCheck Boot 1 is of a dimension that ensures internal and external rotation of the lower leg 50 is limited. The length of the foam wedge 40 extends from the patient's mid-calf to just above the ankle at the ridge 49 of the foam wedge 40, as illustrated in FIG. 9.

It is to be noted that the fiber fill 2 of the HeelCheck Boot 1 comprises any of several materials that provide a soft, pliable, medium-dense texture. The angled or sloped top 41 of the foam wedge 40 is utilized to ensure the patient's heel 52 remains comfortably elevated and exposed to air without hyper-extending the knee joint. By elevating the heel 52, combined with a circular cutout 44, which allows suspension of the heel 52 itself, pressure is offloaded from the heel 52, not just reduced.

The combination of fiber fill 2 and the foam wedge 40 works effectively together to eliminate or significantly reduce any pressure points created by the use of a foam component alone. The foam/fiber combination extends under the ankle and terminates at a point above the patient's heel 52 so the HeelCheck 1 boot is prevented from making direct contact with the patient's heel 52. The HeelCheck 1 boot is constructed with left outer and inner sidewalls 10, 60 and right outer and inner sidewalls 20, 61 designed to support the patient's lower leg and ankle.

The leg support fiber/foam portion extends between the inner and outer sidewalls 10, 60, 20, 61 so as to support the patient's lower leg with the heel elevated well above and out of contact with any bedding or mattress. The heel 52 is completely elevated and thus exposed to air circulation by means of the circular cutout 44.

The boot additionally uses dual types of material for its outer fabric 4 and inner fabric 5 covering. The outer fabric 4 repels any fluid that may come into contact with the HeelCheck 1 boot. An inner fabric 5, which may comprise a brushed micro-suede material, absorbs fluids, including a patient's perspiration, and also is antibacterial.

The disclosed boot is fabricated with left and right “Y-straps” 16, 17, 27, 28 that provide secure upper and lower fastening of the HeelCheck 1 boot as it envelops the patient's foot 51 and lower leg 50. Finally, the HeelCheck 1 boot also uses upper and lower “cross-straps” 30, 31 which keep the patient's foot 51 at a 90 degree angle relative to the lower leg 50 and help eliminate foot drop. By eliminating foot drop, essential blood flow is enhanced and heel cord tightening is eliminated. A 90 degree angle, as measured between the plane of the sole of a patient's foot 51 and his/her lower leg 50 is the optimum foot orientation which allows adequate blood circulation.

While preferred embodiments of the present inventive method have been shown and disclosed herein, it will be obvious to those persons skilled in the art that such embodiments are presented by way of example only, and not as a limitation to the scope of the inventive concept. Numerous variations, changes, and substitutions may occur or be suggested to those skilled in the art without departing from the intent and scope of this inventive concept. Such variations, changes, and substitutions may involve other features which are already known per se and which may be used instead of, in combination with, or in addition to features already disclosed herein.

What is claimed is:

1. A heel and lower leg-supporting medical rehabilitative boot for bed patients, comprising a unitary, foldable body form comprising vie connectingly-stitched sections, the sections consisting of a left sidewall, a right sidewall, a rectangular bottom, an inner flap, and an outer flap, and further comprising:
a) a continuous interior fabric form and a continuous exterior fabric form, each fabric form corresponding to the contours of the five connectingly-stitched sections;
b) a rectangular, sloped foam wedge having a circular cutout at one end; and
c) a left Y-strap, a right Y-strap, a left connector square, a right connector square, a left oblong fastener, an upper cross-strap, a lower cross-strap, and a “Toes” tag; and
d) a means for fastening the left Y-strap, right Y-strap, upper cross-strap, and lower cross-strap onto the exterior surfaces of the rehabilitative hoot.

2. A medical rehabilitative boot as in claim 1, wherein the left sidewall comprises an upper and a lower tube hole and the right sidewall additionally comprises an upper and lower tube hole.

3. A medical rehabilitative boot as in claim 1, wherein the left sidewall, right sidewall, and the inner flap are filled with fiber;

4. A medical rehabilitative boot as in claim 1, wherein the outer flap is tilled with foam material and the bottom section encompasses the sloped foam wedge, further having a quantity of fiber material inserted atop the sloped surface of the foam wedge.

5. A medical rehabilitative boot as in claim 1, wherein the outer fabric comprises a “rip-stop,” woven, nylon-threaded material which is generally water resistant.

6. A medical rehabilitative boot as in claim 1, wherein the inner fabric comprises a brushed micro-suede material which is treated to create anti-bacterial properties.

7. A medical rehabilitative boot as in claim 1, wherein the means of fastening the left Y-strap, right Y-strap, upper cross-strap, and lower cross-strap about the exterior of the rehabilitative boot comprises hook-and-felt fastening systems attached to each of the straps, and attached to corresponding positions on the exterior surfaces of the medical rehabilitative boot.

8. A medical rehabilitative boot as in claim 1, wherein the outer and inner flaps are mutually connectable to each other, the means of fastening comprising opposing components of a hook-and-felt fastening system attached to each of the flaps.

9. A medical rehabilitative boot as in claim 1, wherein a “Toes” tag is attached to the right upper edge of the outer flap.

10. A heel and lower leg-supporting medical rehabilitative boot for bed patients, comprising a unitary, foldable body form comprising five connectingly-stitched sections, said sections consisting of a left sidewall, a right sidewall, a rectangular bottom, an inner flap, and an outer flap, wherein
a) the left sidewall comprises an inner fabric surface and an outer fabric, surface, both surfaces stitched together at their perimeters so as to retain a quantity of fiber fill;
b) the right sidewall comprises an inner fabric surface and an outer fabric surface, both surfaces stitched together at their perimeters so as to retain a quantity of fiber fill;
c) the rear edge of the left sidewall is stitched to an edge of the outer flap and the lower edge of the left sidewall is stitched to the left side of the bottom section;
d) the rear portion of the right sidewall is stitched to an edge of the inner flap and the lower portion of the right sidewall is stitched to the right side of the bottom section;
e) the rectangular bottom comprises an inner fabric surface and an outer fabric surface, both surfaces enveloping a rear-to-front downwardly sloped foam wedge, which wedge manifests a circular cutout at its rear end, and further comprises a quantity of fiber fill atop the foam wedge;
f) the rectangular bottom further comprises a circular arc corresponding to the circular cutout of the foam wedge;
g) the outer flap is attachable to the inner flap, and further, comprises an inner fabric surface and an outer fabric surface, and interiorly contains foam material;
h) the inner flap is attachable to the outer flap, and further, comprises an inner fabric surface and an outer fabric surface, and interiorly contains fiber fill; and
i) a left Y-strap, a right Y-strap, a left connector square, a right connector square, a left oblong fastener, an upper cross-strap, and a lower cross-strap are attached, at a first end, to the exterior surfaces of the medical rehabilitative boot so, as to be connectable, at a second end, to specific locations on the exterior surfaces of the medical rehabilitative boot

11. A medical rehabilitative boot as in claim 10, wherein the left sidewall comprises an upper and a lower tube hole and the right sidewall additionally comprises an upper and lower tube hole.

12. A medical rehabilitative boot as in claim 10, wherein the left sidewall, right sidewall, and the inner flap are filled with fiber;

13. A medical rehabilitative boot as in claim 10, wherein the outer flap is filled with foam material and the bottom section encompasses the sloped foam wedge, further having a quantity of fiber material inserted atop the sloped surface of the foam wedge.

14. A medical rehabilitative boot as in claim 10, wherein the outer fabric of all, sections comprises a “rip-stop,” woven, nylon-threaded material which is generally water resistant.

15. A medical rehabilitative boot as in claim 10, wherein the inner fabric of all sections comprises a brushed micro-suede material which is treated to create anti-bacterial properties.

16. A medical rehabilitative boot as in claim 10, wherein the means of fastening the left Y-strap, right Y-strap, upper cross-strap, and lower cross-strap about the exterior of the rehabilitative boot comprises hook-and-felt hastening systems atₜached to each of the straps, and attached to corresponding positions on the exterior surfaces of the medical rehabilitative boot

17. A medical rehabilitative boot as in claim 10, wherein the means of fastening the outer flap to the inner flap comprises a hook-and-felt fastening system attached to each of the flaps.

18. A medical rehabilitative boot as in claim 10, wherein a “Toes” tag is attached to the right upper edge of the outer flap.

19. A method for the prevention of decubitus ulcers of the heel or heels of a human subject who, due to injury or other difficulties, is confined for any length of time in a supine position upon a bed or other structure, the method comprising the steps of:
providing a boot-like apparatus generally conforming to the shape of the human foot and further, said apparatus having a left sidewall foot support structure, a right sidewall foot support structure, both foot support structures joined to the left and right sides of a lower leg and ankle support structure comprising a rectangular foam block/pad having a sloped upper surface and a circular cutout at one end, upon which a person's lower leg may rest, with the heel freely suspended over the circular cutout, an inner flap, an outer flap, and further having a means of detachably connecting a plurality of strap connectors, the inner flap, and the outer flap about the exterior surfaces of the apparatus; and
adapting the apparatus such that insertion of a subject's lower leg within the apparatus causes the lower leg to rest upon the top surface of said rectangular block with the heel of the same lower leg overhanging the circular open space created by the circular cutout.

20. An apparatus as in the method of claim 19, wherein the left sidewall comprises an upper and a lower tube hole and the right sidewall additionally comprises an upper and lower tube hole.

21. An apparatus as in the method of claim 19, wherein the left sidewall, right sidewall, and the inner flap are filled with fiber;

22. An apparatus as in the method of claim 19, wherein the outer flap is filled with foam material and the bottom section encompasses the sloped foam wedge, further having a quantity of fiber material inserted atop the sloped surface of the foam wedge.

23. An apparatus as in the method of claim 19, wherein the outer fabric of all section s comprises a “rip-stop,” woven, nylon-threaded material which is generally water resistant.

24. An apparatus as in the method of claim 19, wherein the inner fabric of all sections comprises a brushed micro-suede material which is treated to create anti-bacterial properties.

25. An apparatus as in the method, of claim 19, wherein the means of fastening the left Y-strap, right Y-strap, upper cross-strap, and lower cross-strap about the exterior of the rehabilitative boot comprises hook-and-felt fastening systems attached to each of the straps, and attached to corresponding positions on the exterior surfaces of the medical rehabilitative boot

26. An apparatus as in the method of claim 19, wherein the means, of fastening the outer flap to the inner flap comprises a hook-and-felt fastening system attached to each of the flaps.
